(19) United States
(12) Patent Application Publication
(10) Pub. No.: US 2025/0331878 A1
(43) Pub. Date: Oct. 30, 2025

(54) MEDICAL DEVICES AND RELATED SYSTEMS AND METHODS OF USE

(71) Applicant: Boston Scientific Medical Device Limited, Galway (IE)

(72) Inventors: Neeraj GARG, Gurgaon (IN); Amit KUMAR, Noida (IN); Sushil KUMAR, Muzaffarpur (IN); Manoj KUMAR, Gurgaon (IN)

(73) Assignee: Boston Scientific Medical Device Limited, Galway (IE)

(21) Appl. No.: 19/185,338

(22) Filed: Apr. 22, 2025

Related U.S. Application Data
(60) Provisional application No. 63/638,607, filed on Apr. 25, 2024.

(51) Int. Cl. A61B 17/221; A61B 17/00; A61B 90/00

(52) U.S. Cl. CPC A61B 17/221; A61B 90/03; A61B 2017/00367; A61B 2017/00429; A61B 2017/2212; A61B 2090/034

(57) ABSTRACT

In some aspects, a first medical device configured to couple to a second medical device may comprise a central support member configured to be temporarily positioned within a portion of a handle of the second medical device; and a first arm extending outward from the central support member, wherein the first arm extends outside of the portion of the handle when the first medical device is coupled to the second medical device, wherein a first width of the first arm and a first length of the first arm is smaller than a second width of the central support member and a second length of the central support member, respectively. The first medical device may be configured to prevent actuation of a first actuator of the handle when the first medical device is coupled to the second medical device.

### CROSS-REFERENCE TO RELATED APPLICATIONS

This application claims the benefit of priority from U.S. Provisional Application No. 63/638,607, filed on Apr. 25, 2024, which is incorporated by reference herein in its entirety.

### TECHNICAL FIELD

Various aspects of this disclosure relate generally to medical devices, systems, and procedures. Particular aspects relate to medical devices and systems for removing material, e.g., obstructions, from one or more body lumens and/or passageways, among other aspects.

### BACKGROUND

During noninvasive procedures, a physician may use a capture device (e.g., a basket or grasper) to engage objects in a body. For example, in some urology procedures, the physician may advance a sheath into a kidney, extend a basket from the sheath, engage a stone in the kidney with the basket, and retract the basket to capture the stone. Often, a capture device may be deployed through a working channel of an endoscope or other insertable device. For example, an endoscope may be placed in the ureter or kidney, and a capture device may be deployed through a working channel of the endoscope and used to “grab” a stone or other tissue for removal. A plurality of stones and/or stone fragments may need to be removed during the procedure, requiring the basket to be extended numerous times.

### SUMMARY

The present disclosure relates to, among other things, medical devices and systems for removing obstructions from body lumens and/or passageways, and related methods of use. Aspects of this disclosure may decrease the time to remove an obstruction, such as a kidney stone, from a patient's body. Further, for example, aspects of the present disclosure may reduce the level of skill of a practitioner sufficient to complete such obstruction removal procedure, and/or may prevent damage to one or more devices during a procedure. Although primarily discussed in the context of kidney stone removal procedures, any of the systems, devices, and features thereof discussed herein may be used or adapted for use in other parts of the body to remove other materials or obstructions. The medical devices and systems disclosed herein in various examples may include one or more of the features or components described in connection with any of the other examples.

In some aspects, a first medical device configured to couple to a second medical device may comprise a central support member configured to be temporarily positioned within a portion of a handle of a second medical device; and a first arm extending outward from the central support member, wherein the first arm extends outside of the portion of the handle when the first medical device is coupled to the second medical device, wherein a first width of the first arm and a first length of the first arm is smaller than a second width of the central support member and a second length of the central support member, respectively. The first medical device may be configured to prevent actuation of a first actuator of the handle when the first medical device is coupled to the second medical device.

In other aspects, the medical device may include one or more of the following features. The central support member is U-shaped and includes a first wall, a second wall, and a third wall; and the first wall is coupled to the first arm. The second wall includes a first end coupled to the first wall and a second end including a protrusion; and the third wall includes a third end coupled to the first wall and a fourth end including a protrusion. The first arm includes a fifth end coupled to the first wall and a sixth end, and the sixth end is expanded relative to the fifth end. The first arm includes a plurality of grip protrusions extending outward from the first arm, and the first arm is configured to be gripped by a user to facilitate insertion and removal of the first medical device into and out of, respectively, the handle. The second end includes a first curved surface and the fourth end includes a second curved surface, and each of the first curved surface and the second curved surface is angled towards a recess between the second wall and the third wall. The first arm extends outward from the central support member from a first end of the central support member; and the first arm is curved towards a second end of the central support member opposite from the first end. A second arm may extend outward from the first end of the central support member; the second arm may be curved towards the second end of the central support member and extends outward from the central support member in a first direction opposite from a second direction in which the first arm extends outward from the central support member.

In other aspects, the medical device may include one or more of the following features. The first arm may include a first end coupled to the central support member and a second end including a first protrusion, and the first protrusion extends away from the central support member. The second arm may include a third end coupled to the central support member and a fourth end including a second protrusion, and the second protrusion extends away from the central support member and the first protrusion. The first arm and the second arm are substantially aligned. Each of the first arm and the second arm is configured to extend around a radially-outward facing surface, relative to a central longitudinal axis of the handle, of the handle. The first arm extends outward from the central support member from a first side of the central support member; a second arm extends outward from the central support member from a second side of the central support member, wherein the first side is positioned on an opposite side of the central support member from the second side of the central support member; and each of the first arm and the second arm includes a lumen, and each lumen is configured to receive a protrusion of the handle. Each of the first arm and the second arm includes a surface flush with a surface of the central support member. The first medical device is configured to extend around at least half of a circumference of the handle.

In other aspects, a medical device may comprise a handle; a sheath coupled to the handle; and a wire extending through the sheath to a distal portion of the sheath, wherein the wire is coupled to an end-effector at a distal end of the wire. The handle may comprise: a handle body including an exterior portion and an interior portion; an actuator positioned within the handle body; and a biasing member positioned within the handle body, wherein a first end of the biasing member abuts the interior portion of the handle body and a second end of the biasing member abuts the actuator; wherein the wire extends through the biasing member; wherein the actuator is configured to transition between (i) a first position in which the end-effector is covered by the sheath and (ii) a second position in which the end-effector is positioned outside of the sheath; and wherein the biasing member is configured to bias the actuator towards the first position.

In other aspects, the medical device may include one or more of the following features. The interior portion of the handle body may include a first protrusion, and the first protrusion is configured to abut the actuator when the actuator is in the second position and is configured to limit proximal movement of the actuator relative to the handle body. The actuator is positioned within a channel of the handle body extending along a longitudinal axis of the handle body. The interior portion of the handle body includes a second protrusion, the second protrusion is configured to abut the actuator when the actuator is in the first position and is configured to limit the distal movement of the actuator relative to the handle body.

In other aspects, a method may comprise coupling a first medical device to a second medical device including positioning the first medical device within a portion of a handle of the second medical device, wherein the first medical device is configured to prevent actuation of a first actuator of the handle when the first medical device is coupled to the second medical device; positioning a shaft of the second medical device at target anatomy; uncoupling the first medical device from the second medical device including removing the first medical device from the portion of the handle; and actuating the first actuator to deploy an end-effector at a distal end of the second medical device.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. Although endoscopes are referenced herein, reference to endoscopes should not be construed as limiting the possible applications of the disclosed aspects. For example, the disclosed aspects may be used with ureteroscopes, duodenoscopes, bronchoscopes, colonoscopes, catheters, sheaths, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 is a side view of an exemplary medical device, according to aspects of this disclosure.

FIG. 2 is a top view of a portion of the medical device of FIG. 1, according to aspects of this disclosure.

FIG. 3A and 3B are side views of a distal portion of the medical device of FIG. 1 in an actuated state and an unactuated state, respectively, according to aspects of this disclosure.

FIGS. 4A and 4B illustrate a proximal portion of the medical device of FIG. 1 and a second medical device, according to aspects of this disclosure.

FIGS. 5A, 5B, and 5C illustrate side, front and perspective views, respectively, of a medical device, according to aspects of this disclosure.

FIGS. 6A and 6B illustrate front, cross-sectional views of a first medical device coupled to a second medical device, according to aspects of this disclosure.

FIGS. 7A and 7B illustrate a proximal portion of the medical device of FIG. 1 and a second medical device, according to aspects of this disclosure.

FIGS. 8A, 8B, and 8C illustrate front, side and perspective views, respectively, of a medical device, according to aspects of this disclosure.

FIG. 9 illustrates a front, cross-sectional view of a first medical device coupled to a second medical device, according to aspects of this disclosure.

FIGS. 10A and 10B illustrate a proximal portion of the medical device of FIG. 1 and a second medical device, according to aspects of this disclosure.

FIGS. 11A, 11B, and 11C illustrate front, side and perspective views, respectively, of a medical device, according to aspects of this disclosure.

FIG. 12 illustrates a front, cross-sectional view of a first medical device coupled to a second medical device, according to aspects of this disclosure.

FIGS. 13A, 13B, and 13C illustrate an alternative embodiment of a handle of a medical device, with a portion of the handle removed in FIGS. 13B and 13C for illustration purposes only, according to aspects of this disclosure.

FIG. 14 illustrates the assembly of the handle shown in FIGS. 13A-13C, according to aspects of this disclosure.

FIGS. 15A-15C are side, cross-sectional views of various alternative embodiments of handles.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

Aspects of various devices are now described. Some aspects are described with reference to noninvasive procedures, such as urology procedures, wherein a sheath is advanced to a treatment site, and a capture device is extended from the sheath to engage an object at the treatment site. In urology procedures, for example, the sheath may be inserted into the urethra, moved through the bladder and ureter, and advanced into a calyx of a kidney; and the capture device may be extended distally from the sheath and/or a working channel of an endoscope or other device to engage one or more stones and/or stone fragments located in the calyx.

When a user inserts a capture device into a working channel of an endoscope (or other device), there is a potential use error by which a user may open a basked or grasper of the capture device inside the working channel of the endoscope without knowing it. This may be due to a handle actuator of the capture device not including a locking mechanism for preventing the accidental opening of a basket or grasper. If the basket or grasper is opened inside of a working channel, the capture device may damage itself and potentially damage the working channel, and thus impact the performance of both devices during a procedure. For example, if the capture device cannot be safely removed from the working channel due to an unintentional opening of a basket or grasper, then an invasive procedure may be required, even further increasing costs.

In another scenario, when a stone is captured in a grasper of a capturing device, a user may have to maintain a slight pressure on a handle actuator to keep the stone in a captured state in the grasper while pulling the capture device from anatomy and through a working channel. There is potential for the user to inadvertently open the basket inside the working channel or the anatomy without knowing it, and this may cause an unwanted release of a stone during a procedure. This may increase the procedure time, potentially injure the patient, and may cause other complications.

References to a particular type of procedure, such as a urology procedure; capture device, such as a basket or grasper; organ, such as a kidney; and/or object, such as a stone or stone fragment, are provided for convenience and not intended to limit this disclosure. Accordingly, the concepts described herein may be utilized for any analogous device or system.

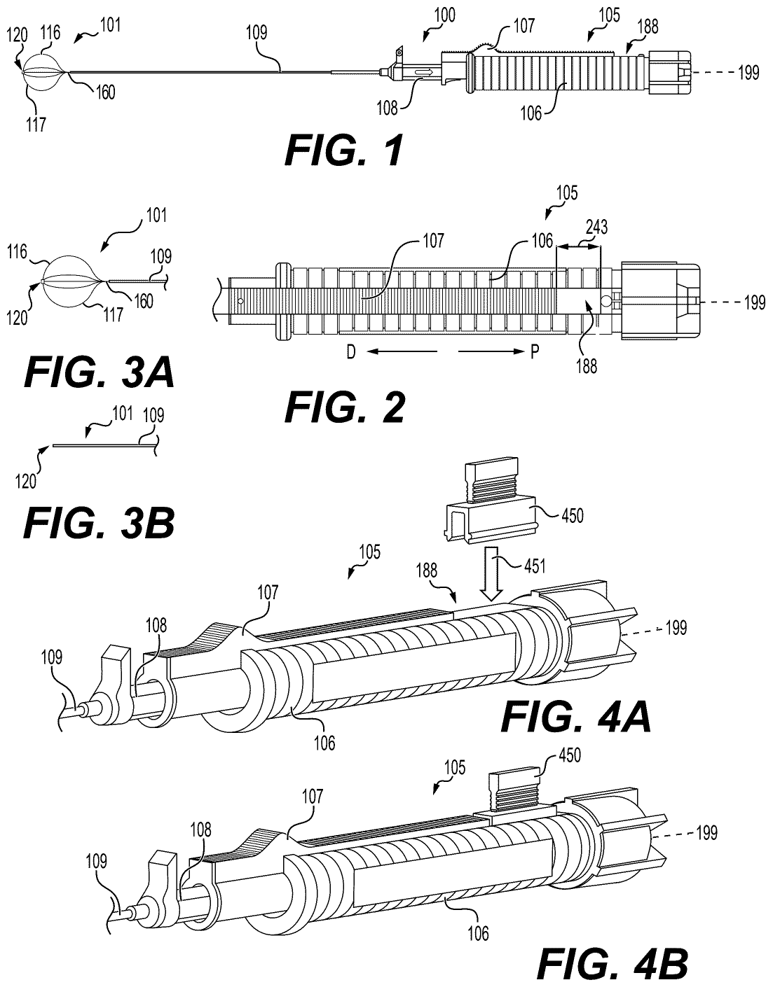

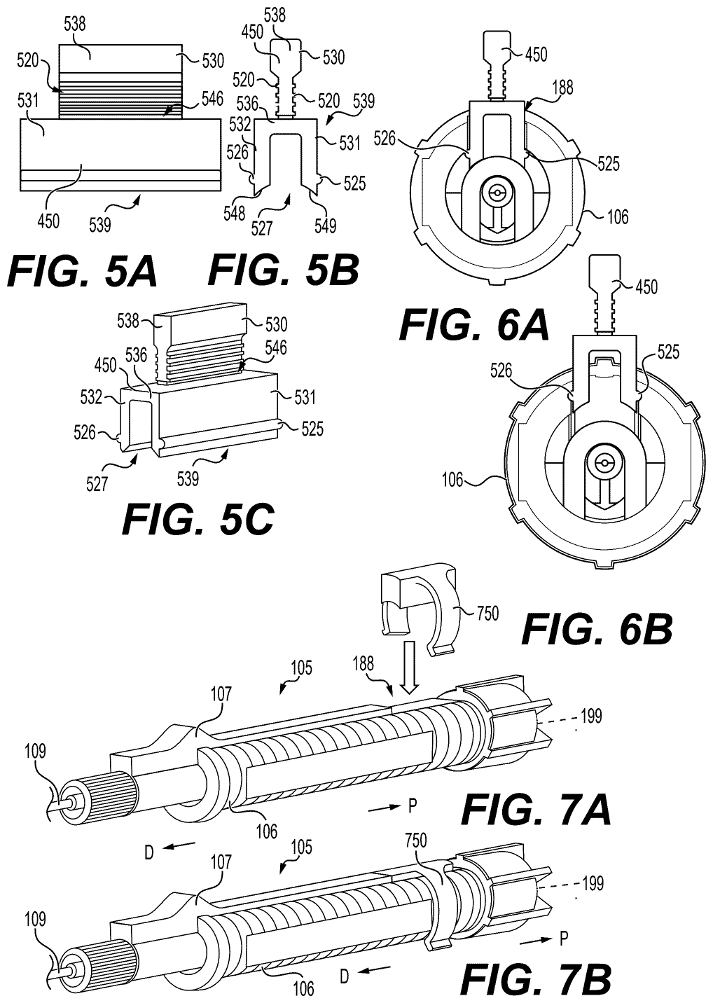

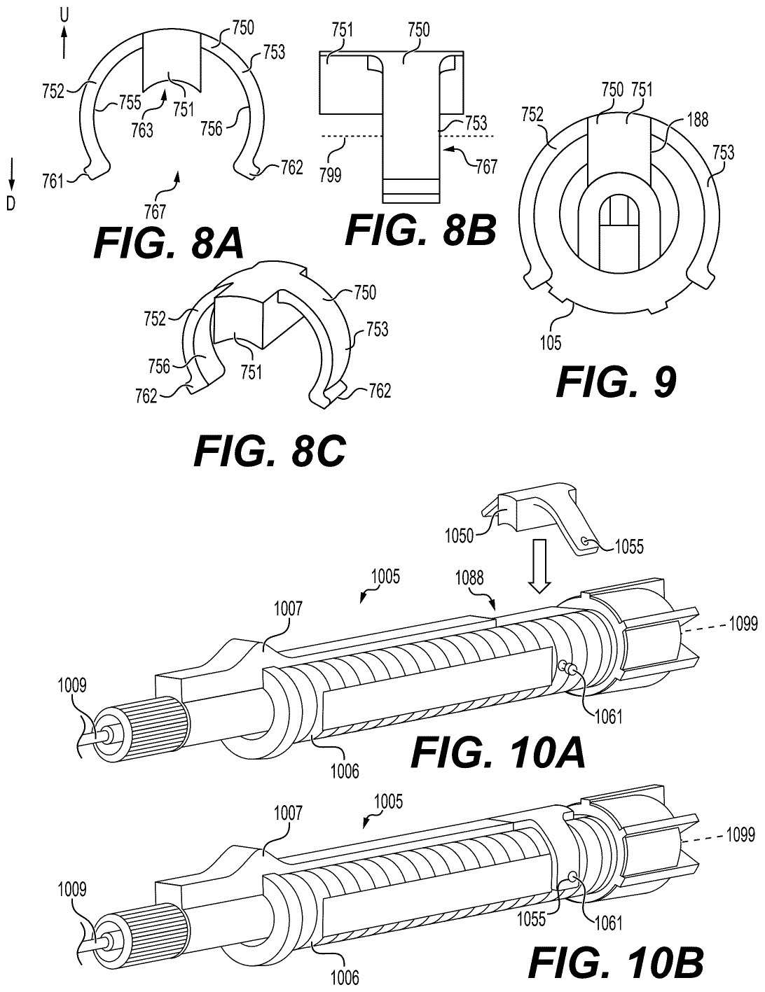

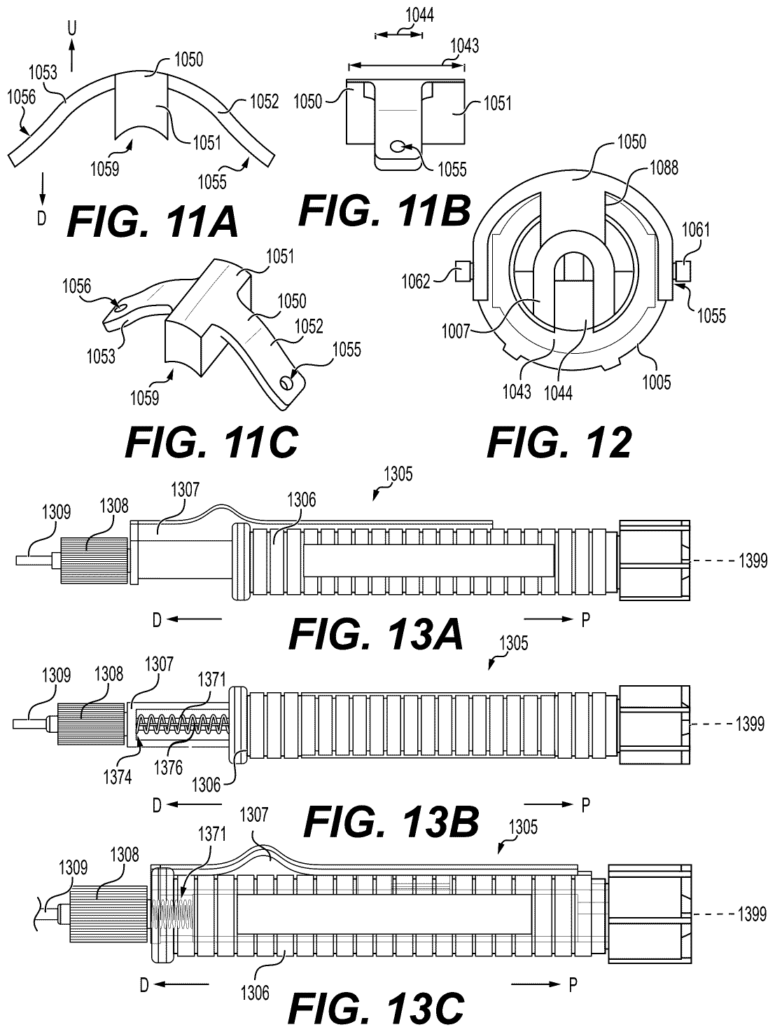

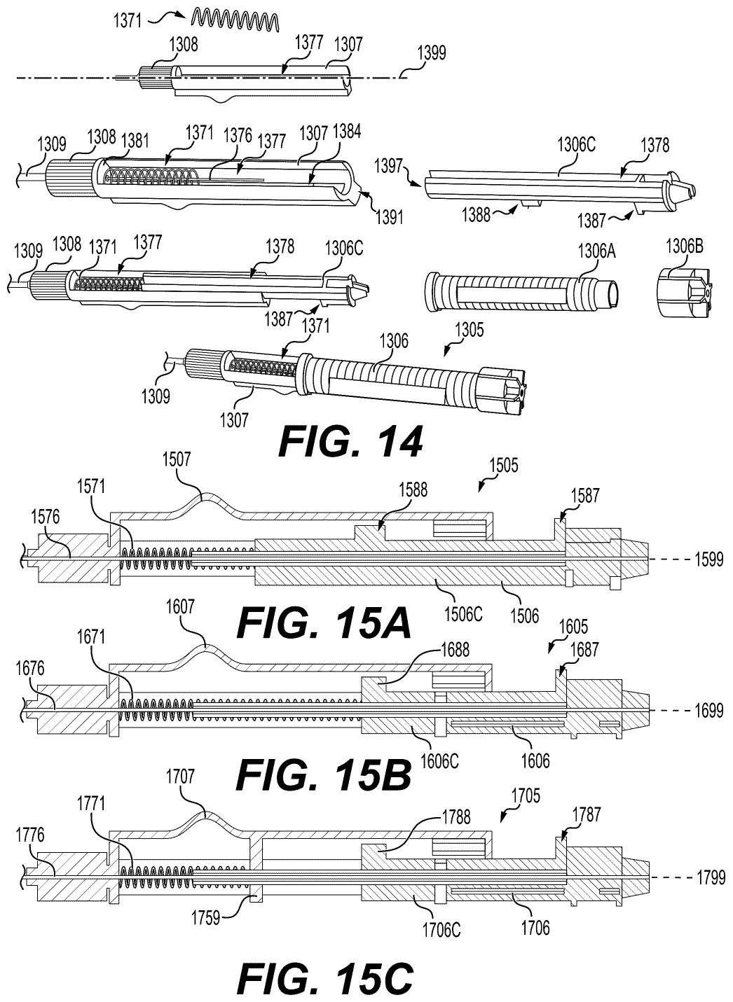

An exemplary medical device 100 is depicted in FIGS. 1 and 2. As shown, device 100 may comprise: a handle 105; an actuator 107 movably mounted to handle 105; and a plunger 108 movably mounted to actuator 107. A shaft or sheath 109 may be attached to plunger 108. A wire 160 may extend distally through handle 105, actuator 107 and plunger 108 from handle 105; and an end-effector 101 may be attached to a distal end of wire 160. Actuator 107 may be operable to extend end-effector 101 distally out of sheath 109 to surround a stone or other material, and retract end-effector 101 proximally into sheath 109 to capture the stone or other material. Specifically, actuator 107 may slide relative to handle body 106 in a proximal or distal direction to move wire 160 proximally or distally through sheath 109, and to deploy end-effector 101 out of sheath (in a deployed configuration) or retract end-effector 101 into sheath 109 (in a retracted configuration).

End-effector 101 is shown as a basket in FIG. 1, however end-effector 101 may be any suitable end-effector 101, such as a grasper, a knife, a stapler, a snare, a snare net, an electrode, and/or any other suitable end-effector 101 known in the art. End-effector 101 may include a plurality of arms 116, 117 extending radially-outward, relative to central longitudinal axis 199, from wire 160. Arms 116, 117 may be evenly spaced around a circumference of wire 160. Each arm 116, 117 may be coupled to each other at a distalmost end 120 of end-effector 101. FIG. 3A illustrates end-effector 101 in a deployed configuration, with each arm 116, 117 positioned at least partially distal from sheath 109. FIG. 3B illustrates end-effector 101 in a retracted configuration with each arm 116, 117 positioned entirely within sheath and covered by 109. Each arm 116, 117 may expand radially-outward when transitioned from the retracted configuration to the expanded configuration.

Handle 105 may be grasped in a hand of a user and/or attached to another medical device. Handle 105 may comprise: a body 106 extending along a central longitudinal axis 199 of device 100. Body 106 is depicted as having a cylindrical shape, although any shape may be used. The exterior surfaces of body 106 may be contoured to define a grip surface. A channel 188 may extend through body 106 parallel to axis 199. Wire 160 may be fixedly coupled to a proximal portion of body 106, and may extend substantially parallel with or coaxial to axis 199.

Actuator 107 may be configured to move sheath 109 in a proximal-distal direction along (e.g., parallel to or coaxial with) longitudinal axis 199. Actuator 107 may be positioned within channel 188 and may be configured to slide within channel 188. Actuator 107 may have a distal or closed position where sheath 109 is positioned over end-effector 101 (e.g. FIG. 3B), and a proximal or open position where sheath 109 is positioned proximal to end-effector 101 and end-effector 101 is deployed outside of sheath 109 (e.g. FIG. 3A). Actuator 107 may be configured to move a set distance 243 within channel 188 between the distal or closed position and the proximal or open position. In some examples, as discussed below, actuator 107 may be spring-biased towards either the distal or closed position or the proximal or open position.

Plunger 108 may be movably mounted to actuator 107 and configured to enable rotation of sheath 109. Wire 160 may be an elongated metallic element, such as a surgical wire, although any type of material may be used. End-effector 101 may be attached to a distal end of wire 160. In FIG. 1, for example, end-effector 101 is a closed-ended basket made of a shape memory metal (e.g., nitinol). End-effector 101 may also be an open-ended grasper, or like capture device. Accordingly, end-effector 101 may be operable between a closed position, wherein end-effector 101 is collapsed inside of sheath 109 (e.g., FIG. 3B); and an open position, wherein end-effector 101 automatically expands after being extended distally out of sheath 109 (e.g., FIG. 3A). As discussed herein above, any type of end-effector 101 may be used with device 100, including any closed-ended configurations (e.g., a basket) or open-ended configurations (e.g., two or more opposing jaws).

FIGS. 4A and 4B illustrate handle 105 of medical device 100 and a retainer device 450, with retainer device 450 spaced from (disconnected from) handle 105 in FIG. 4A and retainer device 450 coupled to handle 105 in FIG. 4B. Retainer device 450 may be designed to prevent or otherwise inhibit the accidental opening or deployment of end-effector 101, and prevent or otherwise inhibit the movement of actuator 107 relative to handle body 106. A user may push retainer device 450 into channel 188 of handle 105, shown by arrow 451 in FIG. 4A. For example, retainer device 450 may be moved in a direction that is substantially perpendicular to axis 199 to insert it into channel 188. Once positioned within channel 188 (shown in FIG. 4B), retainer device 450 may prevent/inhibit movement of actuator 107 within handle 105, and, for example, prevent/inhibit actuator 107 from moving into an open position in which the end-effector 101 is deployed. For example, a user may push retainer device 450 into channel 188, and then move sheath 109 through a working channel of an insertion device (e.g. an endoscope, etc.) to target anatomy. Retainer device 450 may occupy a space of channel 188 that is not occupied by actuator 107 and my inhibit actuator 107 from moving into the space of channel 188 that would be vacant without a presence of retainer device 450. In other words, retainer device 450 may be temporarily inserted into channel 188 during portions of a procedure when accidental deployment of end effector 101 is desired to be prevented.

Retainer device 450 may be removed when actuation of actuator 107 is desired. The user may pull retainer device 450 out of channel 188 and move actuator 107 proximally to deploy end-effector 101 at the target anatomy. With retainer device 450 removed from channel 188, actuator 107 may be free to move proximally into the portion of channel 188 that was previously occupied by retainer device 450.

FIGS. 5A, 5B, and 5C illustrate side, front, and perspective views of retainer device 450. Retainer device 450 may include a first wall 530, and first wall 530 may be coupled at a first end 546 to a U-shaped portion 539 of retainer device 450. U-shaped portion 539 may include a second wall 536 coupled to the first wall 530, a third wall 531 extending from a first end of the second wall 536, and a fourth wall 532 extending from a second end of the second wall 536. First wall 530 may extend from second wall 536 in an opposite direction from third wall 531 and fourth wall 532, and first wall 530 may be coupled to second wall 536 at an approximate longitudinal midpoint of second wall 536 (or at a middle-portion between two ends of second wall 536, as shown in FIG. 5B). Each of first wall 530, third wall 531, and fourth wall 532 may be substantially parallel to each other; and second wall 536 may be substantially perpendicular to first wall 530, third wall 531, and fourth wall 532. In some examples, first wall 530 may have a longitudinal length that is shorter than second wall 536, third wall 531, and fourth wall 532. In some examples, each of second wall 536, third wall 531, and fourth wall 532 may have substantially the same longitudinal length. Retainer device 450 may have a longitudinal length that is approximately the same as distance 243 so as to approximately fully occupy a portion of channel 188 unoccupied by actuator 107. Third wall 531 and fourth wall 532 may each have a curved end 548, 549, respectively, opposite from the end coupled to the second wall 536. Curved ends 548, 549 may approximately face each other and may be angled towards recess 527.

First wall 530 may include an expanded end 538 at an opposite end from the first end 546. Expanded end 538 may be a free end that is not connected to another structure. Grip protrusions 520 may extend outward from first wall 530. Grip protrusions 520 may be configured to facilitate grip or increase friction with a user's fingers and/or thumbs. Expanded end 538 may facilitate grabbing/gripping first wall 530 to position retainer device 450 into and out of channel 188. U-shaped portion 539 may form a recess 527 extending between third wall 531 and fourth wall 532. Each of third wall 531 and fourth wall 532 may include a protrusion 525, 526, respectively, at an end opposite from the end coupled to second wall 536. Each protrusion 525, 526 may extend outward from third wall 531 or fourth wall 532 away from recess 527, such that neither protrusion 525, 526 is positioned within recess 527. Each protrusion 525, 526 may be configured to engage a portion of handle 105 to facilitate coupling retainer device 450 to handle 105, specifically within channel 188.

Retainer device 450 may be made of rubber, for example a unitary rubber component with high shore hardness. For example, retainer device 450 may be made of PELLETHANE 2363, with a shore hardness 50-60D.

FIGS. 6A and 6B illustrate cross-sectional views of handle body 106 with retainer device 450 coupled to handle body 106. The cross-sectional views of handle body 106 in FIGS. 6A and 6B are taken facing proximally along central longitudinal axis 199. One or more sidewalls of handle body 106 may include one or more recesses configured to receive protrusions 525, 526 of retainer device 450. Protrusions 525, 526 may snap into position upon positioning protrusions 525, 526 within such one or more recesses of handle body 106. Protrusions 525, 526 may prevent movement of retainer device 450 out of channel 188 during operation of medical device 100.

FIGS. 7A and 7B illustrate handle 105 of medical device 100 and a retainer device 750, with retainer device 750 spaced from (separated from) handle 105 in FIG. 7A and retainer device 750 coupled to handle 105 in FIG. 7B. Retainer device 750 may be designed to prevent the accidental opening or deployment of end-effector 101, and prevent the movement of actuator 107 relative to handle body 106. A user may push retainer device 750 into channel 188 of handle 105. Once positioned within channel 188 (shown in FIG. 7B), retainer device 750 may prevent movement of actuator 107 within handle 105, and, for example, prevent actuator 107 from moving into an open position in which the end-effector 101 is deployed. For example, a user may push retainer device 750 into channel 188, and then move sheath 109 through a working channel of an insertion device (e.g. an endoscope, etc.) to target anatomy. Similarly to retention device 450, retention device 750 may occupy a portion of channel 188 that is unoccupied by actuator 107 when it is in a distal (unactuated) position, thereby inhibiting proximal movement of actuator 107. Then the user may pull retainer device 750 out of channel 188 and move actuator 107 proximally to deploy end-effector 101 at the target anatomy.

FIGS. 8A, 8B, and 8C illustrate front, side, and perspective views of retainer device 750. Retainer device 750 may include a central support 751 (e.g., a central support member) and two curved arms 752, 753 extending outward from the central support 751. Up and down directional arrows (i.e. U arrow and D arrow) are shown in FIG. 8A to provide a directional reference with regard to retainer device 750. Each arm 752, 753 may extend from an uppermost edge of central support 751 and may extend downward beyond a lowermost edge of central support 751, such that a length of each arm 752, 753 in the up-down direction is longer than a length of central support 751 in an up-down direction. A bottom portion of arm 752 (in the up-down direction) may curve towards arm 753, and a bottom portion of arm 753 (in the up-down direction) may curve towards arm 752, and each arm 752, 753 may, in some examples, have the same longitudinal length. Each of arms 752, 753 may have a convex, arcuate shape. Arm 752 and arm 75 may be substantially aligned.

A protrusion 761 may be positioned at a first end of arm 752 opposite from the end of arm 752 that is coupled to central support 751, and a protrusion 762 may be positioned at a first end of arm 753 opposite from the end of arm 753 that is coupled to central support 751. Each protrusion 761, 762 may be configured to facilitate a user's fingers grasping retainer device 750 and pulling or pushing retainer device 750 off of or onto handle 105, respectively.

In some examples, a radially-inward facing surface 755 of arm 752 may have the same center of curvature as a radially-inward facing surface 756 of arm 753. Central support 751, arm 752, and arm 753 may form a partially open passage 759 configured to receive a portion of handle 105. A central longitudinal axis 799 may extend through a gap 767, and each protrusion 761, 762 may extend radially outward relative to the central longitudinal axis 799. In some examples, the central longitudinal axis 799 may extend through the center of curvature of radially-inward facing surfaces 755, 756. Gap 767 may be formed between the ends of arms 752, 753, and gap 767 may have a width that is smaller than the width of handle 105. Retainer device 750 may be configured to extend around more than half of the circumference of handle 105, and thus may couple to handle 105. A user may remove (uncouple) retainer device 750 from handle 105 by pulling retainer device away from handle 105 (e.g. by pulling on protrusions 761, 762), which may bend arms 752, 753 radially outward, relative to axis 799, and remove (uncouple) central support 751 from channel 188.

Central support 751 may have a longitudinal length extending along central axis 799 that is longer than the longitudinal length, extending along central axis 799, of each arm 752, 753. For example, central support 751 may have a length that is approximately the same as distance 243. Central support 751 may include a curved surface 763 configured to engage a portion of handle 105 within channel 188. Central support 751 may be configured to be temporarily positioned within channel 188, and arms 752, 753 may be configured to extend around a radially-outer surface of handle 105. FIG. 9 illustrates a front, cross-sectional view of retainer device 750 coupled to handle 105, with central support 751 positioned within channel 188 and arms 752, 753 extending around a radially-outer circumference of handle 105. Retainer device 750 may be a plastic molded component, and/or may be made of Acrylonitrile Butadiene Styrene and/or any other suitable material.

FIGS. 10A and 10B illustrates an alternative embodiment of a handle 1005, which may be used instead of handle 105 in medical device 100. Handle 1005 may include an actuator 1007, a handle body 1006, and a channel 1088. A sheath 1009 may be coupled to handle 1005. A central longitudinal axis 1099 may extend through handle 1005. Handle 1005 may include any of the features described herein regarding handle 105. A pair of support protrusions 1061, 1062 may extend radially-outward, relative to axis 1099, from handle body 1006. Support protrusion 1061 may be positioned on an opposite side of handle body 1006 as support protrusion 1062, and support protrusion 1061 may be radially-aligned with support protrusion 1062. Support protrusions 1061, 1062 may be positioned at a proximal portion of handle 1005, and may be ninety degrees offset, relative to axis 1099, from channel 1088.

Handle 1005 is shown spaced (separated) from a retainer device 1050 in FIG. 10A, and retainer device 1050 is coupled to handle 1005 in FIG. 10B. Retainer device 1050 may be designed to prevent the accidental opening or deployment of end-effector 101, and prevent the movement of actuator 1007 relative to handle body 1006. A user may push retainer device 1050 into channel 1088 of handle 1005, and may position support protrusions 1061, 1062 within lumens 1055, 1056 of retainer device 1050. Protrusions 1061, 1062 positioned within lumens 1055, 1056 may prevent retainer device 1050 from moving out of channel 1088. Once positioned within channel 188 (shown in FIG. 7B), retainer device 1050 may prevent movement of actuator 1007 within handle 1005, and, for example, prevent actuator 1007 from moving into an open position in which the end-effector 101 is deployed. Similar to retainer devices 450 and 750, retainer device 1050 may occupy a portion of channel 1088 that is not occupied by actuator 1007 and may thus inhibit actuator 1007 from moving proximally into that portion of channel 1088. For example, a user may push retainer device 1050 into channel 1088, position protrusions 1061, 1062 within lumens 1055, 1056, and then move sheath 109 through a working channel of an insertion device (e.g. an endoscope, etc.) to target anatomy. Then the user may remove protrusions 1061, 1062 from lumens 1055, 1056, pull retainer device 1050 out of channel 188 (thereby uncoupling retainer device 1050 from handle 105) and move actuator 1007 proximally to deploy end-effector 101 at the target anatomy.

FIGS. 11A, 11B, and 11C illustrate front, side, and perspective views, respectively, of retainer device 1050. Retainer device 1050 may include a central support 1051 and two arms 1052, 1053 extending outward from the central support 1051. Each arm 1052, 1053 may be curved and/or flexible and/or bendable. Up and down directional arrows (i.e. U arrow and D arrow) are shown in FIG. 11A to provide a directional reference with regard to retainer device 1050. Each arm 1052, 1053 may extend from an uppermost edge of central support 1051 and may extend downward beyond central support 1051, such that a length of each arm 1052, 1053 in the up-down direction is longer than a length of central support 1051 in an up-down direction (e.g. when arms 1052, 1053 are bended downward). In some examples, each arm 1052, 1053 may have the same longitudinal length. A first lumen 1055 may be positioned at an end of arm 1052 opposite from the end coupled to central support 1051, and a second lumen 1056 may be positioned at an end of arm 1053 opposite from the end coupled to central support 1051. Lumens 1055, 1056 may be configured to receive protrusions 1061, 1062 of handle 1005, to facilitate coupling retainer device 1050 to handle 1005.

Central support 1051 may have a longitudinal length 1043 that is longer than the width 1044 of each arm 1052, 1053. For example, central support 1051 may have a longitudinal length that is approximately the same as a longitudinal length of a portion of channel 1088 that is not occupied by actuator 1007. Central support 1051 may include a curved surface 1059 configured to engage a portion of handle 1005 within channel 1088. Central support 1051 may be configured to be positioned within channel 1088, and arms 1052, 1053 may be configured to extend around a radially-outer surface of handle 1005. Arms 1052 may be flexible and may configured to wrap around a circumference of handle 1005.

FIG. 12 illustrates a front, cross-sectional view of retainer device 1050 coupled to handle 1005, with central support 1051 positioned within channel 1088 and arms 1052, 1053 extending around a radially-outer circumference of handle 105. Protrusions 1061, 1062 of handle 1005 are shown positioned within lumens 1055, 1056 in FIG. 12. Retainer device 1050 may be a plastic molded component, and/or may be made of a rubber, such as a heat-cured high consistency silicone rubber such as a silicone elastomer, and/or any other suitable material. In some examples, retainer device 1050 may be made of the Dow Corning SILASTIC® Q7-4765 Biomedical Grade ETR Elastomer.

The lumens 1055, 1056 in arms 1052, 1053 of retainer device 1050 may facilitate coupling retainer device 1050 to handle 1005, may reduce movement of retainer device 1050 relative to handle 1005 during operation of medical device 100, and may facilitate coupling retainer device 1050 to handle 1005 during a procedure.

FIGS. 13A, 13B, and 13C illustrate side views of an alternative embodiment of a handle 1305. Handle 1305 may have any of the features described herein with relation to any of the other handles 105, 1005. Portions of handle 1305 are shown as transparent and/or removed in FIGS. 13B and 13C to expose interior components of handle 1305. Handle 1305 may include a handle body 1306, an actuator 1307, a plunger 1308, and a central longitudinal axis 1399. A sheath 1309 may be coupled to the plunger 1308 at a distal end of the plunger 1308, and a wire 1376 (or other member) may extend longitudinally through handle body 1306 and be coupled to a proximal end of handle body 1306. A biasing member 1371 may be coupled to plunger 1308 and a distal end 1374 of actuator 1307, and biasing member 1371 may be coupled to a proximal portion of handle body 1306. In some examples, biasing member 1371 may extend between plunger 1308/distal end 1374 of actuator 1307 and the proximal portion of handle body 1306 without being fixedly coupled to either (e.g., may merely abut the relevant portion of handle 1305). Biasing member 1371 may be a spring, a body made of a shape memory material, or other biasing member known in the art. Biasing member 1371 may extend circumferentially around wire 1376, and both biasing member 1371 and wire 1376 may be substantially aligned with central longitudinal axis 1399.

FIG. 13B illustrates handle 1305 in a closed position, with sheath 1309 extending over the end-effector 101 (e.g. as shown in FIG. 3B), such that the end-effector 101 is covered. Actuator 1307 is extended distally from handle body 1306 in the closed position, and biasing member 1371 is in an uncompressed state (or neutral state). Biasing member 1371 may be biased towards the closed position, such that if a user releases actuator 1307 then biasing member 1371 may move actuator 1307 to the closed position. FIG. 13C illustrates handle 1305 in an open position, with end-effector 101 extended distally out of sheath 1309 (e.g. as shown in FIG. 3A). Actuator 1307 is positioned within handle body 1306 and a proximal end of plunger 1308 is adjacent to a distal end of handle body 1306 when actuator is in a fully open position. Actuator 1307 may be more within handle body 1306 in the fully open position compared to the fully closed position. Biasing member 1371 may be in a fully compressed state when actuator 1307 is in a fully open position, and may be biased such that if a user releases actuator 1307 when in a fully open position, actuator 1307 will then transition from the open position to a closed position by moving plunger 1308, actuator 1307, and sheath 1309 distally relative to handle body 1306, and thus moving sheath 1309 over end-effector 101.

FIG. 14 illustrates an exemplary assembly process of handle 1305. Biasing member 1371 may be positioned within a channel 1377 of actuator 1307. Although actuator 1307 is shown including a channel 1377, alternatively actuator 1307 may be cylindrical and include a circular passage that receives biasing member 1371. Biasing member 1371 may then be positioned around wire 1376 within channel 1377. In some examples, biasing member 1371 may be fixedly coupled to a proximal-facing surface 1381 of actuator 1307 at a distal end of actuator 1307, and in other examples biasing member may abut but not be fixedly coupled to the proximal-facing surface 1381. An interior component 1306C of handle body 1306 may then be positioned within channel 1377, proximal of biasing member 1371.

Interior component 1306C may include a longitudinally-extending channel 1378 and two protrusions 1387, 1388 extending radially-outward, relative to central longitudinal axis 1399. First protrusion 1387 may be configured to abut a proximal-most end 1391 of actuator 1307 to limit the proximal movement of actuator 1307 within handle body 1306. Second protrusion 1388 may be configured to be positioned within a recess 1384 within channel 1377. Second protrusion 1388 may be configured to abut a proximal end of recess 1384 to limit the distal movement of actuator 1307 relative to handle body 1306. Interior component 1306C may be configured to fit within channel 1377 of actuator 1307, and a distalmost end 1397 of interior component 1306C may be configured to abut biasing member 1371. In some examples, biasing member 1371 may be fixedly coupled to distalmost end 1397 of interior component 1306C. An exterior component 1306A of handle body 1306 may then be positioned over actuator 1307 and interior component 1306C, and a proximal cap 1306B of handle body 1306 may be coupled to a proximal end of exterior component 1306A and/or a proximal end of interior component 1306C. Once assembled, a user may move actuator 1307 proximally or distally relative to handle body 1306, and sheath 1309 may move proximally or distally relative to end-effector 101 to transition between open and closed configurations.

FIGS. 15A, 15B, and 15C illustrate alternative embodiments of handles 1505, 1605, 1705 in side, cross-sectional views. Each of handles 1505, 1605, 1705 may have any of the features described herein in relation to any of the other handles 105, 1005, 1305; and may function in the same manner as handle 1305. Handle 1505 may include a handle body 1506 with interior component 1506C, an actuator 1507, a central longitudinal axis 1599, and a wire 1576 extending longitudinally through handle 1505. Interior component 1506C may include protrusions 1587, 1588. Biasing member 1571 may be positioned within handle body 1506 in the same manner described hereinabove in relation to biasing member 1371. Interior component 1506C may have a shorter longitudinal length compared to interior component 1306C to accommodate a biasing member 1571 with a longer longitudinal length compared to biasing member 1371.

Handle 1605 may include a handle body 1606 with interior component 1606C, an actuator 1607, a central longitudinal axis 1699, and a wire 1676 extending longitudinally through handle 1605. Interior component 1606C may include protrusions 1687, 1688. Biasing member 1671 may be positioned within handle body 1606 in the same manner described hereinabove in relation to biasing member 1371. Interior component 1606C may have a shorter longitudinal length compared to interior component 1306C to accommodate a biasing member 1671 with a longer longitudinal length compared to biasing member 1371. Protrusion 1688 may be positioned at the distalmost end of interior component 1606C.

Handle 1705 may include a handle body 1706 with interior component 1706C, an actuator 1707, a central longitudinal axis 1799, and a wire 1776 extending longitudinally through handle 1705. Interior component 1706C may include protrusions 1787, 1788. Biasing member 1771 may be positioned within handle body 1706 in the same manner described hereinabove in relation to biasing member 1371. Interior component 1706C may have a shorter longitudinal length compared to interior component 1306C. Actuator 1707 may include an interior wall 1759 configured to abut biasing member 1771, and to allow for a shorter longitudinal length of biasing member 1771. Protrusion 1788 may be positioned at the distalmost end of interior component 1706C.

In operation, medical device 100 may be used to remove material from a patient's body, and may facilitate a variety of medical procedures such as kidney stone removal procedures. A user may first insert an endoscope or other insertion device into a patient and position a distal end of endoscope at target anatomy. For medical devices 100 that incorporate handle 105, the user may then position one of retainer devices 450, 750, 1050 into the channel 188 of handle body 106 to prevent end-effector 101 from transitioning from a closed position to an open position. For medical devices 100 that incorporate one of handles 1305, 1505, 1605, 1705, the user does not need to couple a retainer device 450, 750, 1050 to the handle 1305, 1505, 1605, 1705 since the biasing member 1371, 1571, 1671, 1771 provides a bias towards the closed position and prevents end-effector 101 from transitioning to an open configuration when the user is not actuating actuator 1307, 1507, 1607, 1707.

The user may then position medical device 100 within a lumen/working channel of the endoscope and move distal end 120 of the medical device 100 out of a distal opening of the lumen/working channel. The user may then transition medical device 100 from a closed configuration to an open configuration, either by removing the retainer device 450, 750, 1050 from handle 105 and actuating actuator 107; or by actuating actuator 1307, 1507, 1607, 1707 (e.g. by moving actuator 107, 1307, 1507, 1607, 1707 proximally relative to handle body 106, 1306, 1506, 1606, 1706). The user may then remove medical device 100 from lumen/working channel of the endoscope, either by first coupling retainer device 450, 750, 1050 to handle 105 and then pulling medical device 100 proximally through the lumen/working channel, or by pulling medical device 100 proximally through the lumen/working channel for medical devices with handles 1305, 1505, 1605, 1705.

The medical devices, systems, and methods discussed in this disclosure may facilitate removal of kidney stones or other tissue from a patient's body, may increase the amount of material that can be removed from a patient during a single procedure, may reduce the procedure time, may reduce the risk of damaging medical device 100 during a procedure, and may reduce the risk of injury to the patient. For example, the devices in this disclosure may prevent damage to medical device 100 during a procedure, thus reducing the need for repeated entries into the patient's body with different medical devices during a single procedure.

Any of the medical devices 100, 450, 750, 1050 described herein may be made of any suitable material, such as plastic, PEBAX, or any other suitable material. In any of the above-described examples, medical devices 100, 450, 750, 1050 may include one or more components that are metallic, a polymer, machined, formed, stamped, insert molded, or any combination thereof. Any aspect(s) of any one of the above-described examples of medical device 100, 450, 750, 1050 may be incorporated into any of the other examples of medical devices 100, 450, 750, 1050 described herein. Moreover, any of the handles 105, 1005, 1305, 1505, 1605, 1705 described herein may be incorporated into medical device 100.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A first medical device configured to couple to a second medical device, comprising:
a central support member configured to be temporarily positioned within a portion of a handle of a second medical device; and
a first arm extending outward from the central support member, wherein the first arm extends outside of the portion of the handle when the first medical device is coupled to the second medical device, wherein a first width of the first arm and a first length of the first arm is smaller than a second width of the central support member and a second length of the central support member, respectively;
wherein the first medical device is configured to prevent actuation of a first actuator of the handle when the first medical device is coupled to the second medical device.

2. The first medical device of claim 1, wherein the central support member is U-shaped and includes a first wall, a second wall, and a third wall;
wherein the first wall is coupled to the first arm.

3. The first medical device of claim 2, wherein the second wall includes a first end coupled to the first wall and a second end including a protrusion; and
wherein the third wall includes a third end coupled to the first wall and a fourth end including a protrusion.

4. The first medical device of claim 3, wherein the first arm includes a fifth end coupled to the first wall and a sixth end, wherein the sixth end is expanded relative to the fifth end.

5. The first medical device of claim 4, wherein the first arm includes a plurality of grip protrusions extending outward from the first arm, and
wherein the first arm is configured to be gripped by a user to facilitate insertion and removal of the first medical device into and out of, respectively, the handle.

6. The first medical device of claim 5, wherein the second end includes a first curved surface and the fourth end includes a second curved surface, wherein each of the first curved surface and the second curved surface is angled towards a recess between the second wall and the third wall.

7. The first medical device of claim 1, wherein the first arm extends outward from the central support member from a first end of the central support member; and
wherein the first arm is curved towards a second end of the central support member opposite from the first end.

8. The first medical device of claim 7, further comprising a second arm extending outward from the first end of the central support member; wherein the second arm is curved towards the second end of the central support member and extends outward from the central support member in a first direction opposite from a second direction in which the first arm extends outward from the central support member.

9. The first medical device of claim 8, wherein the first arm includes a first end coupled to the central support member and a second end including a first protrusion, wherein the first protrusion extends away from the central support member.

10. The first medical device of claim 9, wherein the second arm includes a third end coupled to the central support member and a fourth end including a second protrusion, wherein the second protrusion extends away from the central support member and the first protrusion.

11. The first medical device of claim 10, wherein the first arm and the second arm are substantially aligned.

12. The first medical device of claim 11, wherein each of the first arm and the second arm is configured to extend around a radially-outward facing surface, relative to a central longitudinal axis of the handle, of the handle.

13. The first medical device of claim 1, wherein the first arm extends outward from the central support member from a first side of the central support member; and
wherein a second arm extends outward from the central support member from a second side of the central support member, wherein the first side is positioned on an opposite side of the central support member from the second side of the central support member; and
wherein each of the first arm and the second arm includes a lumen, and each lumen is configured to receive a protrusion of the handle.

14. The first medical device of claim 13, wherein each of the first arm and the second arm includes a surface flush with a surface of the central support member.

15. The first medical device of claim 14, wherein the first medical device is configured to extend around at least half of a circumference of the handle.

16. A medical device comprising:
a handle;
a sheath coupled to the handle; and
a wire extending through the sheath to a distal portion of the sheath, wherein the wire is coupled to an end-effector at a distal end of the wire;
wherein the handle comprises:
a handle body including an exterior portion and an interior portion;
an actuator positioned within the handle body; and
a biasing member positioned within the handle body, wherein a first end of the biasing member abuts the interior portion of the handle body and a second end of the biasing member abuts the actuator;
wherein the wire extends through the biasing member;
wherein the actuator is configured to transition between (i) a first position in which the end-effector is covered by the sheath and (ii) a second position in which the end-effector is positioned outside of the sheath; and
wherein the biasing member is configured to bias the actuator towards the first position.

17. The medical device of claim 16, wherein the interior portion of the handle body includes a first protrusion, wherein the first protrusion is configured to abut the actuator when the actuator is in the second position and is configured to limit proximal movement of the actuator relative to the handle body.

18. The medical device of claim 17, wherein the actuator is positioned within a channel of the handle body extending along a longitudinal axis of the handle body.

19. The medical device of claim 18, wherein the interior portion of the handle body includes a second protrusion, wherein the second protrusion is configured to abut the actuator when the actuator is in the first position and is configured to limit distal movement of the actuator relative to the handle body.

20. A method comprising:
coupling a first medical device to a second medical device including positioning the first medical device within a portion of a handle of the second medical device, wherein the first medical device is configured to prevent actuation of a first actuator of the handle when the first medical device is coupled to the second medical device;
positioning a shaft of the second medical device at target anatomy;
uncoupling the first medical device from the second medical device including removing the first medical device from the portion of the handle; and
actuating the first actuator to deploy an end-effector at a distal end of the second medical device.
